(19) United States
(12) Patent Application Publication
(10) Pub. No.: US 2008/0039379 A1
(43) Pub. Date: Feb. 14, 2008

(54) Compositions Comprising Gastrin Compounds and Their Use in Diabetes

(75) Inventor: Antonio Cruz, Toronto (CA)

Correspondence Address: MINTZ, LEVIN, COHN, FERRIS, GLOVSKY;AND POPEO, P.C., ONE FINANCIAL CENTER, BOSTON, MA, 02111, US

(73) Assignee: WARATAH PHARMACEUTICALS, INC., Toronto, ON (CA)

(21) Appl. No.: 10/558,523

(22) PCT Filed: May 27, 2004
(86) PCT No.: PCT/CA04/00769

§ 371 Date: Jun. 23, 2006

(30) Foreign Application Priority Data
May 27, 2003 (US) PCT/US03/16660

(51) Int. Cl. A61K 38/16; A61K 38/10; A61P 3/08

(52) U.S. Cl. 514/12; 514/13; 514/2

(57) ABSTRACT

The invention relates generally to novel compositions and methods comprising a gastrin compound. The compositions and methods provide beneficial effects, in particular sustained beneficial effects, in the treatment of diabetes.

### FIELD OF THE INVENTION

The invention relates generally to compositions and methods comprising a gastrin compound, and uses thereof.

### BACKGROUND OF THE INVENTION

Gastrin and other growth factors have been implicated in the development of fetal pancreas (Brand and Fuller, J. Biol. Chem. 263:5341-5347). Gastrin is transiently expressed in the fetus in the pancreatic islets and its expression is confined to the period when protodifferentiated islet precursors form differentiated islets. While the significance of pancreatic gastrin expression in islets is unknown, elevated pancreatic gastrin has been observed with nesidioblastosis. In particular, an abnormal persistence of pancreatic gastrin has been documented in a case of infantile nesidioblastosis (Hollande et al, Gastroenterology, 71:251-262, 1976) and hypergastrinemia caused by gastrin-expressing islet cell tumors. Atrophic gastritis has also been associated with nesidioblastosis similar to that seen in differentiating fetal islets (Sacchi et al, Virchows Archiv B, 48:261-276, 1985). However, in neither observation was a casual relationship established between the nesidiobastosis and gastrin stimulation.

The citation of any reference herein is not an admission that such reference is available as prior art to the instant invention.

### SUMMARY OF THE INVENTION

The invention provides a composition, in particular a pharmaceutical composition, comprising one or more gastrin compound that provides beneficial effects in the treatment of diabetes and its complications.

In an aspect the invention provides a pharmaceutical composition, comprising one or more gastrin compound that provides beneficial effects, in particular sustained beneficial effects, following treatment. The beneficial effects provided by a composition of the invention can include increased absorption, distribution, metabolism and/or elimination of a gastrin compound. A composition can have increased bioavailability (absorbed more rapidly and to a higher degree) or provide enhanced therapeutic effects, in particular sustained beneficial effects.

The invention also provides a pharmaceutical composition intended for administration to a patient to provide beneficial effects, in particular sustained beneficial effects, comprising a gastrin compound, optionally together with pharmaceutically acceptable carriers, excipients, or vehicles.

The invention also provides a pharmaceutical composition for the treatment of a disease or condition comprising a therapeutically effective amount of a gastrin compound to provide a sustained beneficial effect in a pharmaceutically acceptable carrier, excipient, or vehicle.

In an embodiment, a pharmaceutical composition comprising a gastrin compound is provided which has been adapted for administration to a subject to provide sustained beneficial effects to treat a condition or disease. In a preferred embodiment, the composition is in a form such that administration to a subject results in blood glucose levels that are about normal that persist in the subject for a sustained period of time after cessation of treatment.

In another embodiment, the invention relates to a liquid drug formulation comprising a gastrin compound or pharmaceutically acceptable salts thereof, and to lyophilized drug formulations that can be reconstituted to provide suspensions that are stable and suitable for parenteral administration.

In a particular embodiment, the invention relates to an aqueous composition comprising a gastrin compound adapted to provide sustained beneficial effects. The invention also provides a drug comprising an aqueous formulation of a gastrin compound that provides sustained beneficial effects, or pharmaceutically acceptable salts thereof with at least one solubilizer.

The present invention is directed to compositions comprising a gastrin compound that provides beneficial effects, in particular sustained beneficial effects, in the treatment of a condition or disease in particular, diabetes.

In another aspect, the invention features a composition comprising a gastrin compound in a dosage effective for inducing proliferation of islet precursor cells into an increased amount of mature insulin secreting cells, in particular for a sustained period following administration of the gastrin compound. Proliferation of islet precursor cells may be induced ex vivo or in viva. The composition can be in a dosage effective for inducing differentiation of an islet precursor cell into a mature insulin secreting cell. The composition can be in a pharmaceutically acceptable carrier, excipeint, or vehicle.

The invention additionally provides a method of preparing a stable pharmaceutical composition comprising one or more gastrin compound adapted to provide beneficial effects, preferably sustained beneficial effects, following treatment. A method can comprise mixing one or more gastrin compound, and a pharmaceutically acceptable carrier, excipient, or vehicle, in particular, a pharmaceutically acceptable carrier, excipient, or vehicle effective to physically stabilize the gastrin compound(s). After compositions have been prepared, they can be placed in an appropriate container and labelled for treatment of an indicated condition. For administration of a composition of the invention, such labelling would include amount, frequency, and method of administration.

The invention also contemplates the use of a composition comprising at least one gastrin compound for the preparation of medicaments for preventing and/or treating conditions and/or diseases. The invention additionally provides uses of a pharmaceutical composition of the invention in the preparation of medicaments for the prevention and/or treatment of conditions and/or diseases. The medicaments provide beneficial effects, preferably sustained beneficial effects following treatment.

The invention provides a method for treating and/or preventing a condition and/or disease in a subject comprising administering to the subject a therapeutically effective amount of one or more gastrin compound to provide beneficial effects. In an aspect the invention provides a treatment which results in sustained beneficial effects following treatment.

The invention has particular applications in preventing and/or treating diabetes. Thus, the invention relates to a method of treatment comprising administering a therapeutically effective amount of one or more gastrin compound which upon administration to a subject with symptoms of diabetes produces beneficial effects, preferably sustained beneficial effects. In an embodiment, sustained beneficial effects are evidenced by one or more of the following: (a) an increase in C-peptide production, (b) an increase in pancreatic insulin production, and/or (c) about normal blood glucose levels.

In an embodiment, the invention provides a method for preventing and/or treating Type I or Type II diabetes comprising administering a therapeutically effective amount of a composition of the invention.

In a further embodiment, the invention provides a method for amelioriating progression of a condition and/or disease or obtaining a less severe stage of a condition and/or disease in a person suffering from Type I or Type II diabetes comprising administering a therapeutically effective amount of a composition of the invention.

The invention relates to a method of delaying the progression of impaired glucose tolerance or non-insulin requiring Type II diabetes to insulin requiring Type II diabetes comprising administering a therapeutically effective amount of a composition of the invention.

The invention also relates to a method of increasing the insulin synthesis capability of a subject comprising administering a therapeutically effective amount of a composition of the invention.

In embodiments of methods of the invention the subject is not treated with insulin.

The invention provides a kit comprising one or more gastrin compound or a pharmaceutical composition of the invention. In an aspect, the invention provides a kit for preventing and/or treating diabetes, containing a composition comprising one or more gastrin compound, a container, and instructions for use. The composition of the kit can further comprise a pharmaceutically acceptable carrier, excipient, or vehicle.

These and other aspects, features, and advantages of the present invention should be apparent to those skilled in the art from the following drawing and detailed description.

### DESCRIPTION OF THE DRAWINGS

The invention will be better understood with reference to the drawings in which:

FIG. 1 is a bar graph showing the results of treating NOD mice with recent-onset diabetes with either E1 (1 μug/Kg/day I.P. for 14 days) or G1 (3 μg/Kg/day I.P. for 14 days). Fasting blood glucose levels (mM) at day 0 and day 35 after diabetes onset (FBG>6.6 mM) are shown.

FIG. 2 is a bar graph showing the results of treating NOD mice with recent-onset diabetes with either E1 or G1 as described for FIG. 1. Pancreatic insulin content (μg/pancreas) is shown for vehicle alone at onset of diabetes and at day 35. Treatment stopped after day 14.

FIG. 3 is a line graph showing the results of monitoring NOD mice with recent-onset diabetes for eight weeks, including an initial 18 day period of treatment with gastrin. Fasting glucose levels (mM) at week 0 and each week thereafter are shown.

FIG. 4 is a graph showing treatment with G1 decreases fasting blood glucose levels in chronically diabetic insulin-dependent NOD mice and prevents death 14 days after cessation of insulin therapy.

### DETAILED DESCRIPTION OF EMBODIMENTS

### Glossary

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

Compounds described herein can contain one or more asymmetric centers and may give rise to enantiomers, diasteriomers, and other stereoisomeric forms which may be defined in terms of absolute stereochemistry as (R)- or (S)-. Therefore, the invention includes all such possible diasteriomers and enantiomers as well as their racemic and optically pure forms. Optically active (RX and (S)-isomers may be prepared using chiral synthons or chiral reagents, or resolved using conventional techniques. When the compounds described herein contain centers of geometric asymmetry, and unless specified otherwise, it is intended that the compounds include both E and A geometric isomers. All tautomeric forms are intended to be included within the scope of the invention.

The terms “subject”, “individual”, “recipient” or “patient” refer to an animal including a warm-blooded animal such as a mammal, which is afflicted with or suspected of having or being pre-disposed to a disease or a condition described herein. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. The methods herein for use on subjects/individuals/patients contemplate prophylactic as well as curative use. Typical subjects for treatment include persons susceptible to, suffering from or that have suffered a condition or disease described herein. In embodiments of the invention a subject is diabetic.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc include but are not limited to saline, buffered saline, dextrose, water, glycerol, ethanol, and combinations thereof. The use of such media and agents for an active substance is well known in the art.

“Pharmaceutically acceptable salt(s),” includes salts of acidic or basic groups which may be present in the compounds suitable for use in the present invention. Examples of pharmaceutically acceptable salts include sodium, calcium, ammonium, ferric hydroxides, isopropylamine, triethylamine, 2-ethylamine, 2-ethylamino, ethanol, histidine, procarine, and potassium salts of carboxylic acid groups and hydrochloride salts of amino groups. Other pharmaceutically acceptable salts of amino groups are hydrobromide, sulfate, hydrogen sulfate, phosphate, acetate, oxalic, hydrogen phosphate, dihydrogen phosphate, acetate, succinate, citrate, tartrate, lactate, mandelate, methanesulfonate (mesylate) and p-toluenesulfonate (tosylate) salts.

The term “preventing and/or treating” refers to the administration to a subject of a composition of the invention either before or after onset of a condition or disease. A treatment may be either performed in an acute or chronic way.

A “beneficial effect” refers to an effect of a gastrin compound or composition thereof including favorable pharmacological and/or therapeutic effects, and improved pharmacokinetic properties and biological activity. In embodiments of the invention, beneficial effects include but are not limited to the following: reduced or absent islet inflammation, decreased or prevention of disease progression, increased survival, or treatment or reversal of a disease or condition.

In an embodiment, the beneficial effects can be evidenced in diabetes by one or more of the following: (a) a reduction in fasting blood glucose levels, in particular when blood glucose levels are greater than 7-10 mM; (b) reduction in glycosylated haemoglobin; (c) increase in serum insulin concentration; (d) an increase in pancreatic insulin production or content; and/or (e) prevention of disease progression. In a particular embodiment, the beneficial effects comprise (a), (b) and (c), or (a), (c), and (d).

In a preferred embodiment, the beneficial effect is a “sustained beneficial effect” where the beneficial effect is sustained for a prolonged period of time after termination of treatment. A beneficial effect may be sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, or 2 weeks to 18 months following treatment. The period of time a beneficial effect is sustained may correlate with the duration and timing of the treatment. A subject may be treated continuously for about or at least about 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, periodically or continuously. A sustained beneficial effect may manifest as one or more of increased C-peptide production, increased pancreatic insulin production or concentration, and about normal or low blood glucose levels for a prolonged period following treatment.

The beneficial effect may be a statistically significant effect in terms of statistical analysis of an effect of a gastrin compound versus the effects without the compound. “Statistically significant” or “significantly different” effects or levels may represent levels that are higher or lower than a standard. In embodiments of the invention, the difference may be 1.5, 2, 3, 4, 5, or 6 times higher or lower compared with the effect obtained without a gastrin compound.

“Therapeutically effective amount” relates to the amount or dose of an active compound (e.g. gastrin compound) or composition of the invention that will lead to one or more desired beneficial effects, in particular, one or more sustained beneficial effects. A therapeutically effective amount of a substance can vary according to factors such as the disease state, age, sex, and weight of the individual, and the ability of the substance to elicit a desired response in the individual. Dosage regima may be adjusted to provide the optimum therapeutic response (e.g. sustained beneficial effects). For example, several divided doses may be administered daily or the dose may be proportionally reduced as indicated by the exigencies of the therapeutic situation.

A “native-sequence polypeptide” or “a native polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95%, 98%, or 99% amino acid sequence identity to the sequences identified in any of SEQ ID NOs. 1 through 5. Such variants include, for example, polypeptides wherein one or more amino acid residues are added to, or deleted from, the N- or C-terminus of the full-length or mature sequences of SEQ ID NOs: 1 through 5 including variants from other species, but excludes a native-sequence polypeptide.

Percent identity of two amino, acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST programs are publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An “analog” refers to a polypeptide wherein one or more amino acid residues of a parent polypeptide have been substituted by another amino acid residue, one or more amino acid residues of a parent polypeptide have been inverted, one or more amino acid residues of the parent polypeptide have been deleted, and/or one or more amino acid residues have been added to the parent peptide. Such an addition, substitution, deletion, and/or inversion may be at either of the N-terminal or C-terminal end or within the parent polypeptide, or a combination thereof.

Mutations may be introduced into a polypeptide by standard methods, such as site-directed mutagenesis and PCR-mediated mutagenesis. Conservative substitutions can be made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed.

A “derivative” refers to a polypeptide in which one or more of the amino acid residues of a parent polypeptide have been chemically modified. A chemical modification includes adding chemical moieties, creating new bonds, and removing chemical moieties. A polypeptide may be chemically modified, for example, by alkylation, acylation, glycosylation, pegylation, ester formation, deamidation, or amide formation.

A “chimeric polypeptide” comprises all or part (preferably biologically active) of a selected polypeptide operably linked to a heterologous polypeptide (i.e. a polypeptide other than the selected polypeptide). Within the fusion protein, the term “operably linked” is intended to indicate that a selected polypeptide and the heterologous polypeptide are fused in-frame to each other. The heterologous polypeptide can be fused to the N-terminus or C-terminus of a selected polypeptide. Chimeric and fusion proteins can be produced by standard recombinant DNA techniques.

A “gastrin compound” is understood to refer to any compound, including peptides and non-peptide compounds, which fully or partially, directly or indirectly, potentiate, induce, mimic, or otherwise enhance the activity of a gastrin or a gastrin/CCK receptor. In particular, a gastrin compound can be used which fully or partially associates and/or activates a gastrin/CCK receptor. A gastrin/CCK receptor includes receptors that associate with a gastrin.

In some applications of the invention, a gastrin compound may be a ligand that associates, binds to, interacts with or stimulates a gastrin/CCK receptor. A gastrin compound may be selected that is a peptide or non-peptide small molecule that has a suitable IC₅₀, for example an IC₅₀ of about ˜0.7 nM, as measured by methods known in the art (see Singh et al (1995) J. Biol. Chem. 270: 8429-8438, and Kopin et al (1995) J. Biol. Chem. 270: 5019-5023 describing in vitro cell growth assays, and receptor binding assays as described in Singh et al (1995) J. Biol. Chem. 270: 8429-8438, and Kopin et al (1995) J. Biol. Chem. 270: 5019-5023).

A “gastrin compound” can include native-sequence or synthetic gastrin polypeptides, fragments, analogs (e.g. muteins), derivatives, isoforms, variants, chimeric polypeptides, polypeptides with sequence identity, peptidomimetics, and pharmaceutically acceptable salts thereof, and active metabolites and prodrugs. In particular the term includes the various forms of gastrin, preprogastrin, progastrin, such as gastrin 34 (big gastrin), gastrin 17 (little gastrin), gastrin 8 (mini gastrin), pentagastrin, tetragastrin and fragments, analogs, and derivatives thereof.

Sequences for gastrins including big gastrin-34 (Bonato et al, 1986, Life Science 39:959) and small gastrin-17 (Bentley et al (1966) Nature 209:583) are shown in SEQ ID NOs. 1-5. Big gastrin-34 is essentially an extension of an amino acid sequence at the N-terminal end of small gastrin-17. Big gastrin is cleaved in vivo to release gastrin-17. Glp at the N-terminal end is pyroglutamate, which is a naturally cyclized form of glutamate. In various embodiments, where cysteine or lysine are added to a terminus of gastrin having a pyroglutamate, the pyroglutamate is replaced with a glutamate, or the pyroglutamate is deleted. Further, each of a gastrin 34 and gastrin-17 can be used having a methionine or a leucine at position 15, as shown in SEQ ID NOs: 1-4 herein. A gastrin compound can be sulfated or nonsulfated. [See J. H. Walsh, “Gastrin” in Gut Peptides: Biochemistry and Physiology, ed. J. H. Walsh and G. J. Dockray, Raven Press Ltd., New York, 1994 for a review of Gastrin.]

A gastrin compound also includes active analogs, fragments and other modifications, which for example share amino acid sequence identity with an endogenous mammalian gastrin or native-sequence gastrin, for example, share 60%, 70%, 80%, 90%, 95%, 98%, or 99% sequence identity.

Examples of gastrin compounds that may be used in the present invention include the compounds disclosed in U.S. Pat. No. 6,288,301. In some applications of the invention, a gastrin compound may be selected that is a peptide or non-peptide agonist or partial agonist of the gastrin receptor such as A71378 (Lin et al., Am. J. Physiol. 258 (4 Pt 1): G648, 1990). In other applications of the invention, a gastrin compound may be a gastrin/CCK receptor ligand including but not limited to gastrin compounds described herein, or a cholecystokinin (CCK) such as CCK 58, CCK 33, CCK 22, CCK 12 and CCK 8; and the like.

Gastrin compounds also include substances that increase the secretion of endogenous gastrins, cholecystokinins or similarly active peptides from sites of tissue storage. Examples of these are the gastric releasing peptide, omeprazole which inhibits gastric acid secretion and increases plasma gastrin levels, soya bean trypsin inhibitor which increases CCK stimulation, and gastrin releasing peptide, which stimulates gastrin secretion without binding to gastrin receptors.

Gastrin compounds may be prepared using conventional processes. For example, small forms of gastrin such as gastrin 17 are economically prepared by peptide synthesis, and the synthetic peptides are commercially available. In particular, gastrin compounds may be synthesized by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. B. Wansch, Vol. 15 I and II, Thieme, Stuttgart). The synthesis may be performed using manual procedures or by automation. Automated synthesis may be carried out, for example, using an Applied Biosystems 431A peptide synthesizer (Perlin Elmer).

Gastrin compounds can be prepared by recombinant methods well known to those skilled in the art. Thus, the invention contemplates the use of a nucleotide sequence encoding a gastrin compound and optionally a regulatory element, and a host cell comprising the nucleotide sequence for the preparation of a gastrin compound.

Gastrin compounds may also be obtained from commercial sources. For example, synthetic human gastrin 17 with methionine or leucine at position 15 are available from Bachem AG, Bubendorf, (Switzerland), and from Research Plus Inc (New Jersey, USA).

“Host cells” comprising a nucleotide sequence of a gastrin compound include a wide variety of prokaryotic and eukaryotic host cells. For example, the polypeptides may be expressed in bacterial cells such as E. coli, Bacillus, or Streptomyces, insect cells (using baculovirus), yeast cells, or mammalian cells. Other suitable host cells can be found in Goeddel, Gene Expression Technology: Methods in Enzymology 185, Academic Press, San Diego, Calif. (1991). A host cell may also be chosen which modulates the expression of an inserted nucletotide sequence, or modifies (e.g. glycosylation or phosphorylation) and processes (e.g., cleaves) the polypeptide in a desired fashion. Host systems or cell lines may be selected which have specific and characteristic mechanisms for post-translational processing and modification of proteins. For long-term high-yield stable expression of the protein, cell lines and host systems which stably express the gene product may be engineered.

“Regulatory element” refers to a genetic element or elements having a regulatory role in gene expression, for example, promoters or enhancers, Large numbers of suitable vectors and promoters are known to those of skill in the art and are commercially available for generating recombinant constructs encoding a gastrin compound. The following vectors are provided by way of example. Bacterial: pBs, phagescript, PsiX174, pbluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTL pSG (Stratagene) pSVK3, pBPV, pMSG, pSVL (Pharmacia). As defined herein “operably linked” means that an isolated polynucleotide and a regulatory element are situated within a vector or cell in such a way that the polypeptide is expressed by a host cell which has been transformed (transfected) with the ligated polynucleotide/regulatory element sequence. A regulatory element can be a constitutive or induced transcriptional regulatory region, for example, a transcriptional regulatory region from an insulin gene that is induced by increasing intracellular glucose concentrations.

“Conditions and/or diseases”, “condition(s)” and “disease(s)” include diabetes and its complications. The term “diabetes” as used herein means any manifested symptoms of diabetes in any mammal including experimental animal models, and including human forms such as type I and type II diabetes, early stage diabetes, and a pre-diabetic condition characterized by mildly decreased insulin or mildly elevated blood glucose levels. A “pre-diabetic condition” describes a subject demonstrating a symptom in terms of insulin or glucose level, and/or demonstrating a susceptibilty to diabetes or a related condition due to family history, genetic predisposition, or obesity in the case of type II diabetes, and includes a subject who has previously had diabetes or a related condition and is subject to risk of recurrence.

“Insulinotropic activity” refers to an ability of a substance to stimulate insulin secretion in response to elevated glucose levels, to produce or increase glucose uptake by cells, and decreased serum glucose or blood glucose levels. Methods known in the art can be employed to assay for insulinotropic activity. For example, in vitro and in vivo methods may be used that measure gastrin receptor binding activity, receptor activation (see the methods described in EP 619,322 to Gelfand et al and U.S. Pat. No. 5,120,712), and insulin or C-peptide levels. Compounds or compositions described herein have insulinotropic activity if islet cells secrete insulin in the presence of the compounds or compositions above background levels or levels in the absence of the gastrin compounds or compositions.

“Islet neogenesis” means formation of new pluripotent pancreatic precursor cells, pancreatic islet precursor cells, or beta cells by proliferation and differentiation, which may or may not have the characteristics of stem cells which have the ability to reproduce in an unlimited manner.

### Compositions and Methods

The invention is related to compositions and methods that utilize one or more gastrin compound to provide beneficial effects, in particular enhanced beneficial effects, more particularly sustained beneficial effects.

In an embodiment, where the disease or condition is diabetes, sustained beneficial effects of a composition or treatment of the invention can manifest as one or more of the following:
a) An increase in pancreatic insulin levels relative to the levels measured in the absence of a gastrin compound after administration to a subject with symptoms of diabetes. Preferably the compounds induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in pancreatic insulin levels in a subject.
b) A reduction or an absence of symptoms of islet inflammation after administration to a subject with symptoms of diabetes.
c) A decrease in blood glucose levels relative to the levels measured in the absence of a gastrin compound in subjects with symptoms of diabetes. Preferably, the compounds induce at least about a 2%, 5%, 10%, 15%, 20%, 30%, 40%, 50%, 60%, 70%, 80%, or 90% decrease in blood glucose levels. Most preferably, the compounds yield blood glucose levels about or close to the levels common in a normal subject.
d) An improvement in glucose tolerance. In particular, at least about a 5-95%, 10-90%, 10-80%, 10-70%, 10-60%, improvement in glucose tolerance.
e) An increase in C-peptide levels relative to the levels measured in the absence of gastrin compounds in subjects with symptoms of diabetes. Preferably, the compounds induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in C-peptide levels.
f) Maintenance of blood glucose levels at about normal for a prolonged period of time, in particular for at least 1 week, 3 weeks, 4 weeks, 5 weeks, 6 weeks, 8 weeks, 10 weeks, 12 weeks, 14 weeks, 16 weeks, 20 weeks, 24 weeks, 30 weeks, 40 weeks, 52 weeks, or 78 weeks, more particularly, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, or 2 weeks to 18 months.
g) A reduction, prevention, or slowing of the rate of disease progression in a subject with diabetes.
h) A reduction or prevention of the development of severe hyperglycemia and ketoacidosis with symptoms of diabetes.
i) An increase in survival in a subject with symptoms of diabetes.
j) A decrease in requirement for insulin injection/intake by at least 10-90%, 10-80%, 10-70%, 10-60%, 10-50%, 10-40%; 10-30%, or 10-20%.

One or more of these beneficial effects can be demonstrated in a diabetic subject or disease model, for example, a non-obese (NOD) mouse with symptoms of diabetes.

A gastrin compound may be selected for particular applications in the present invention based on one or more of the following characteristics: ability to bind to a gastrin receptor, ability to initiate a signal transduction pathway resulting in proliferation and/or differentiation of beta cells or insulinotropic activity; ability to reduce glucose levels, insulinotropic activity; stimulation of beta cell proliferation/differentiation; and/or, an in vivo half-life of at least about 5 minutes to 24 hours, preferably 2 to 10 hours or 2 to 8 hours in humans using conventional methods.

Pharmaceutical compositions and methods of the invention can be selected that have sustained beneficial effects, preferably statistically significant sustained beneficial effects. In an embodiment, a pharmaceutical composition with statistically significant sustained beneficial effects is provided comprising a gastrin compound selected from the group consisting of gastrin 17 and analogs and derivatives thereof, preferably synthetic human gastrin I having 17 amino acid residues with a Leu residue at amino acid position 15. In a particular embodiment, a pharmaceutical composition with statistically significant beneficial effects is provided comprising gastrin-17(leu).

The invention contemplates the use of a composition of the invention for preventing, and/or ameliorating disease severity, disease symptoms, and/or periodicity of recurrence of a condition and/or disease. The invention also contemplates preventing and/or treating, in mammals, conditions and/or diseases using the compositions or treatments of the invention. In particular, the present invention provides improved methods and compositions for use of a gastrin compound for sustained treatment of diabetes. The present invention in an embodiment provides a composition comprising a gastrin compound that achieves greater efficacy, potency, and utility. The greater efficacy can be shown by improving glucose tolerance in severe diabetes with treatment resulting in sustained improvement of blood glucose after ceasing treatment and also in recent onset diabetes. An improvement in glucose tolerance may also be observed with the compositions described herein using lower doses of gastrin, i.e. doses below 1-50 μg/kg body weight, in particular, 1-30 μg/kg body weight.

Greater efficacy and potency of a gastrin treatment of the invention improves the therapeutic ratio of treatment, reducing untoward side effects and toxicity. The methods of the invention also enhance utility improving long-standing diabetes even when treatment is begun long after the completion of β cell destruction.

While not wishing to be bound by a particular mechanism, improvement in glucose tolerance after treatment with gastrin may result from β cell regeneration and concomitant increased β cell mass. Histological analysis can show treatment with gastrin stimulates β cell regeneration with an increase in the β cell mass as measured morphometrically. This can be confirmed biochemically by an increase in pancreatic insulin content. The increased β cell mass can also result in increased secretion of insulin into the bloodstream which can be shown by increased circulating C peptide in plasma.

Prolonged efficacious islet cell neogenesis can be achieved in accordance with the invention following administration of a gastrin compound or composition of the invention. The gastrin compound or composition can be administered in vivo to provide for proliferation and/or differentiation of islet cells in a subject or it can be administered ex vivo to cells for transplantation. A gastrin compound can be introduced to cells using methods known to a person skilled in the art including recombinant techniques. For example, a chimeric insulin promoter-gastrin fusion gene may be introduced in vivo or ex vivo to pancreatic cells to express one or more gastrin compound.

The invention relates to a method for expanding and differentiating stem cells or progenitor cells into insulin secreting cells comprising contacting the stem cells or progenitor cells with a therapeutically effective amount of a gastrin compound or a composition of the invention or sufficient amounts of a gastrin compound or a composition to expand and differentiate stem cells or progenitor cells. The stem cells may be obtained from pancreatic islets, umbilical cords, embryos, or stem cell lines. The amount and duration of expansion and differentiation is significantly different compared with that achieved in the absence of the gastrin compounds or composition. In an embodiment, the stem cells or progenitor cells are contacted with the gastrin compounds or composition in culture. In another embodiment, the stem cells or progenitor cells are contacted with the gastrin compounds or composition in a subject. The gastrin compounds or composition may be administered to a subject before, during, or after implantation of stem cells in the subject to expand and differentiate the stem cells in the subject for a prolonged period. The method may additionally comprise administering an immunosuppressive agent.

In an aspect, the invention provides a method for treating diabetes mellitus by providing a composition comprising a gastrin compound in an amount sufficient to effect differentiation of pancreatic islet precursor cells to mature insulin-secreting cells for a prolonged period following administration. A gastrin compound in the composition can be administered systemically, in particular by injection, preferably intravenously, in a physiologically acceptable carrier. Alternatively, a gastrin compound can be expressed in situ, and pancreatic islet precursor cells are transformed either ex vivo or in vivo with one or more nucleic acid encoding a gastrin compound in an expression construct vector that provides for expression of the compound in the cells. A nucleic acid encoding a gastrin compound can be contained in an expression construct that may include a preprogastrin peptide precursor coding sequence, which following expression is processed to gastrin. The expression construct can include regulatory elements.

The invention also relates to inducing islet neogenesis in a subject comprising contacting islet precursor cells with a gastrin compound, or composition of the invention in a sufficient amount to increase and prolong proliferation of islet precursor cells in the subject thereby inducing islet neogenesis. In an aspect, the invention provides a method for stimulating prolonged beta cell proliferation in a subject comprising administering a therapeutically effective amount of a gastrin compound or composition of the invention. In an embodiment, the invention provides a method for increasing the number and/or size of beta cells in a subject for a prolonged period comprising administering a therapeutically effective amount of a gastrin compound or a composition of the invention.

Regenerative differentiation of pluripotent pancreatic precursor cells, for example, pancreatic ductal cells, into mature insulin-secreting cells for a prolonged period can be obtained with the gastrin compounds, compositions and methods described herein for treatment of diabetes mellitus, particularly juvenile onset diabetes, and by therapeutic administration of gastrin compounds or compositions which are provided for systemic administration, or for in situ expression within the pancreas.

The invention provides methods for treating diabetes mellitus in a patient in need thereof by administering a composition comprising a gastrin compound in an amount sufficient to effect prolonged differentiation of the patients pancreatic islet precursor cells to mature insulin-secreting cells and/or to stimulate insulin synthesis in existing islet cells. The composition can be administered systemically or expressed in situ by host cells containing a nucleic acid construct in an expression vector wherein the nucleic acid construct comprises a coding sequence for a gastrin compound, together with transcriptional and translational regulatory elements functional in pancreatic islet precursor cells.

In an aspect, the invention provides a method for treating diabetes mellitus in a patient in need thereof which includes administering to the individual a composition that provides a gastrin compound in a dose sufficient to effect prolonged differentiation of pancreatic islet precursor cells to mature insulin-secreting cells. In another aspect, the invention provides a method for treating insulin dependent diabetes, especially Type I or juvenile diabetes mellitus, comprising administering, preferably systemically, a differentiation regenerative amount of a gastrin compound to a diabetic mammal, to stimulate islet neogenesis resulting in an increase in the number of functional glucose responsive insulin secreting β cells in the pancreas for a prolonged period following administration.

The invention contemplates a method of expanding a functional beta cell mass of pancreatic islet transplants in a diabetic patient for a prolonged period, the method comprising administering to the patient a therapeutically effective amount of a gastrin compound, or a composition of the invention.

The invention in an embodiment provides a method for preventing and/or treating diabetes, the method comprising administering to a mammal in need thereof a composition comprising a gastrin compound in an amount sufficient to increase the number of pancreatic insulin secreting β cells in the mammal for a prolonged period following administration, thereby preventing and/or treating the diabetes. The composition is administered systemically. The mammal is a diabetic mammal, for example, the mammal has been diabetic for an extent of 1% of the lifespan of the mammal. The gastrin compound is provided in an amount sufficient to induce differentiation of the pancreatic islet precursor cells into glucose responsive insulin secreting islet cells for a prolonged period.

Another embodiment of the invention provides a method for preventing and/or treating diabetes, the method comprising administering to a mammal in need thereof a composition comprising a gastrin compound in an amount sufficient to increase the amount and duration of proliferation of islet precursor cells in pancreatic tissue for a prolonged period following administration, thereby preventing and/or treating the diabetes.

In another aspect, the invention provides a method for preventing and/or treating diabetes, the method comprising administering to a mammal in need thereof a composition comprising a gastrin compound in an amount sufficient to increase the number of pancreatic insulin secreting β cells in the mammal for a prolonged period following administration; and determining the amount of islet neogenesis, thereby preventing and/or treating the diabetes. The amount of islet neogenesis may be measured by one or more of the following parameters: blood glucose, serum glucose, blood glycosylated hemoglobin, pancreatic β cell mass, serum insulin, and pancreatic insulin content. Administering the composition reduces blood glucose compared to blood glucose assayed prior to administering the composition. Glycosylated hemoglobin concentration is reduced for a prolonged period compared to glycosylated hemoglobin concentration in the mammal assayed prior to administering the composition. Serum insulin concentration is increased for a prolonged period compared to serum insulin concentration in the mammal assayed prior to administering the composition. Pancreatic insulin concentration is increased for a prolonged period compared to pancreatic insulin concentration in the mammal assayed prior to administering the composition.

In a further aspect, the invention provides a method for inducing pancreatic islet neogenesis in a mammal, the method comprising administering to the mammal a composition comprising a gastrin compound, in an amount sufficient to increase the amount and duration of proliferation of islet precursor cells in pancreatic tissue for a prolonged period following administration, thereby inducing pancreatic islet neogenesis. The plurality of cells can be multicellular. The plurality of cells are delivered systemically to the mammal.

In a still further aspect, the invention provides a method for inducing islet neogenesis therapy in a cell of an animal for a prolonged period, comprising contacting the cell with a nucleic acid sequence encoding a gastrin compound operably linked to a regulatory element, for example, an insulin promoter receptor ligand, for example, a metallothionein promoter. For example, the cell is a germ cell, or the cell is an autologous cell cultured ex vivo.

The invention contemplates cell based treatment methods using a gastrin compound of the invention, or compositions of the invention. Thus, the invention contemplates methods comprising treating cells, or treating explanted pancreatic tissue of a mammal with a gastrin compound or composition of the invention and introducing the treated cells or pancreatic tissue to the mammal to provide beneficial effects, in particular sustained beneficial effects. See PCT/CA03/33595 for a description of general culture and cell based treatment methods.

A method for treating a subject with a condition or disease described herein comprises contacting ex vivo a plurality of cells with a gastrin compound, or a composition of the invention, optionally culturing the cells, and administering the cells to the subject in need thereof to provide beneficial effects, in particular sustained beneficial effects.

In embodiments of cell based therapeutic methods the cells are pancreatic ductal cells and the amount of compounds or compositions used in the methods are generally effective to increase the amount of insulin secreting cells in the subject for a prolonged period. The cells may be autologous (i.e. from the same subject), or may be from another individual of the same species, or from a different species.

The invention also contemplates a method for treating diabetes in a subject comprising transplanting a pancreatic islet preparation into the subject and administering a therapeutically effective amount of a gastrin compound, or a composition of the invention to provide beneficial effects, in particular sustained beneficial effects.

The invention also relates to a method for sustaining islet cells or precursor cells in culture comprising culturing the cells in the presence of a gastrin compound or a composition of the invention in an amount sufficient to sustain the cells in culture. The cells may be sustained in culture for a significantly longer period of time compared with cells cultured in the absence of the compounds or composition. Culturing cells in the presence of a gastrin compound or a composition of the invention will be particularly useful in preparing and maintaining cells intended for transplantation.

Also provided are methods and compositions for treating diabetes in a patient in need thereof by implanting into a diabetic patient pancreatic islet cells that have been exposed in culture to a sufficient amount of a gastrin compound to increase the number of pancreatic beta cells in the islets for a prolonged period; optionally the population of pancreatic beta cells can be grown in culture for a time sufficient to expand the population of β-cells prior to transplantation.

Another embodiment of the invention provides a method for treating diabetes, the method comprising: contacting ex vivo a plurality of cells with a composition comprising a gastrin compound in an amount sufficient to increase proliferation of islet precursor cells and the amount of insulin secreting islet cells; and administering the contacted plurality of cells to a mammal in need thereof to produce a beneficial effect, in particular a sustained beneficial effect. The cells can be autologous. The composition is provided in an amount sufficient to effect differentiation of stem cells, for example, to effect differentiation of pancreatic islet precursor cells in pancreatic tissue into mature insulin secreting islet cells. The composition is provided in an amount sufficient to increase proliferation of pancreatic islet stem cells, for example, of pancreatic islet precursor cells for a prolonged period. Stem cells can be obtained either from a pancreatic tissue or from a non-pancreatic tissue, such as liver or bone marrow.

The invention provides a method of treating a condition or disease comprising administering a gastrin compound or composition of the invention with a plurality of cells to a subject in need thereof to thereby produce a beneficial effect, preferably a sustained beneficial effect. In an aspect, the invention provides a method for expanding and differentiating stem cells, in a diabetic recipient of the cells, into insulin secreting cells, the method comprising implanting the cells in the recipient, and administering a composition containing an effective dose of a gastrin compound to produce a beneficial effect, in particular a sustained beneficial effect. For example, the implanted cells are obtained from a human, for example, are obtained from human pancreatic islets, human liver, human bone marrow, human umbilical cord, or human embryos. Implanting the cells into the recipient may be by a route such as injecting directly into an organ, for example, into the pancreas, the kidney, or the liver. Alternatively, implanting the cells may be administering by intravenous injection, for example, into the portal vein or into the hepatic vein. In certain embodiments, prior to implanting the cells are treated ex vivo with a composition comprising a gastrin compound.

The present invention also includes methods of using the compositions of the invention in combination with one or more additional therapeutic agents including without limitation immunosuppressive agents (e.g. rapamycin, cyclosporine, ISAtx247, and FK506), antiobesity agents, antidiabetic agents, appetite regulating drugs, antihypertensive agents, agents for the treatment and/or prevention of complications resulting from or associated with a condition or disease, in particular diabetes and obesity, anti-nausea, anti-headache medications, and general medications that treat or prevent side effects

The invention also contemplates the use of a composition comprising at least one gastrin compound for the preparation of a medicament providing beneficial effects, preferably sustained beneficial effects in treating a condition or disease.

In an embodiment, the invention relates to the use of a therapeutically effective amount of at least one gastrin compound for preparation of a medicament for providing beneficial effects, preferably sustained beneficial effects, in treating a condition or disease.

In an embodiment the invention provides the use of a gastrin compound for the preparation of a medicament for increase preferably prolonged increase) of the number and/or size of beta cells in a subject after treatment.

In another embodiment the invention provides the use of a gastrin compound for the preparation of a medicament for stimulation (preferably prolonged stimulation) of beta cell proliferation after treatment.

In a still further embodiment the invention provides the use of a gastrin compound for the preparation of a medicament for prolonged or sustained treatment of Type I or Type II diabetes.

The invention additionally provides uses of a gastrin compound or a pharmaceutical composition of the invention in the preparation of medicaments for beneficial effects, preferably sustained beneficial effects, in the treatment of diseases and conditions.

Therapeutic efficacy and toxicity of compositions and methods or the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

The methods of the invention may further comprise measuring one or more of the following markers: blood glucose, serum glucose, blood glycosylated haemoglobin, pancreatic beta cell mass, serum insulin, pancreatic insulin levels, morphometrically determined beta cell mass, amount of insulin secreting cells, and glucose responsiveness of insulin secreting cells.

### Administration

A gastrin compound and compositions of the present invention can be administered by any means that produce contact of the active agent(s) with the agent's sites of action in the body of a subject or patient to produce a beneficial effect, in particular a sustained beneficial effect. The active ingredients can be administered simultaneously or sequentially and in any order at different points in time, to provide the desired beneficial effects, in particular sustained beneficial effects. A gastrin compound and composition of the invention can be formulated for sustained release, for delivery locally or systemically. It lies within the capability of a skilled physician or veterinarian to select a form and route of administration that optimizes the effects of the compositions and treatments of the present invention to provide beneficial effects, in particular sustained beneficial effects.

Modes of parenteral administration include, but are not limited to, transdermal, intramuscular, intraperitoneal, intravenous, subcutaneous, intranasal, and oral routes. The compounds may be administered by any convenient route, for example, by infusion or bolus injection, by absorption through epithelial or mucocutaneous linings (e.g. oral mucosa, rectal and intestinal mucosa, etc.), and may be administered together with other biologically active agents. A preferred route of administration is systemic, for example, by subcutaneous injection. For parenteral administration, the compounds and compositions described herein may be combined with saline, PBS, or other suitable buffer, at an appropriate pH. A sustained release formulation can also be used for either or both therapeutic agents.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

An amount of a therapeutic of the invention which will be effective in the treatment of a particular condition or disorder to provide effects, in particular sustained beneficial effects, will depend on the nature of the condition or disorder, and can be determined by standard clinical techniques. The precise dose to be employed in the formulation will also depend on the route of administration, and the seriousness of the condition or disorder, and should be decided according to the judgement of the practitioner and each patient's circumstances. Routine determinations of blood levels of insulin or C peptide, and of fasting levels of glucose or glucose challenges, are determined by one of ordinary skill in the art.

Suitable dosage ranges for administration are particularly selected to provide beneficial effects, in particular sustained beneficial effects. The dosage ranges are generally about 0.01 micrograms to about 500 micrograms of a gastrin compound per kilogram body weight per day, for example, about 0.01 micrograms to about 1 micrograms/kg, about 0.1 micrograms/kg to about 10 micrograms/kg, or about 1 microgram/kg to about 50 micrograms/kg.

In another aspect the invention provides a pharmaceutical composition comprising 0.1 to 30, 0.1 to 40, 0.1 to 50, and 0.1 to 60 micrograms/kg/day gastrin compound.

In particular embodiments of the invention providing sustained beneficial effects, the dosage range for administration of a gastrin compound is 1-30 micrograms/kg body weight, in particular 3-30 micrograms/kg body weight, more particularly 5-20 micrograms/kg body weight.

A composition or treatment of the invention may comprise a unit dosage of at least one gastrin compound to provide beneficial effects, in particular sustained beneficial effects. A “unit dosage” refers to a unitary i.e. a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

In an aspect, a pharmaceutical composition is provided comprising a therapeutically effective suboptimal dosage of a gastrin compound that is effective at decreasing or reducing glucose levels for a sustained period or increasing beta cell proliferation or differentiation following treatment.

In another aspect, an improved pharmaceutical composition is provided comprising therapeutically effective suboptimal amounts of a gastrin compound in a form for chronic or acute therapy of a disease or condition, in particular diabetes.

In an aspect the invention provides a pharmaceutical composition comprising 30-3000, 100-3000, 100-6000, 1000-6000, 2000-6000, and 3000-6000 micrograms gastrin compound per single unit.

A composition or formulation of the invention may be administered to a subject for about or at least about 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, or 2 weeks to 18 months, periodically or continuously. A composition of the invention may be administered one or more times per day, in particular 1 or 2 times per day.

The compositions of the present invention or fractions thereof typically comprise suitable pharmaceutically acceptable carriers, excipients, and vehicles selected based on the intended form of administration, and consistent with conventional pharmaceutical practices.

Suitable pharmaceutical carriers, excipients, and vehicles are described in the standard text, Remington's Pharmaceutical Sciences, Mack Publishing Company. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

The composition can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

In an aspect of the invention a pharmaceutical composition has a pH from about 7 to 10.

Formulations for parenteral administration of a composition of the invention may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

In an embodiment, a composition herein is formulated in accordance with routine procedures as a pharmaceutical composition adapted for subcutaneous or intravenous administration to human beings to provide a beneficial effect, in particular a sustained beneficial effect. Typically, compositions for subcutaneous or intravenous administration are solutions in sterile isotonic aqueous buffer. Where necessary, the composition may also include a solubilizing agent and a local anesthetic to ameliorate pain at the site of the injection. Generally, the ingredients are supplied either separately or mixed together in unit dosage form, for example, as a dry, lyophilized powder or water-free concentrate in a hermetically sealed container such as an ampoule or sachette, for example, indicating the quantity of active agent. Where the composition is to be administered by infusion, an ampoule of sterile water or saline for injection can be provided so that the ingredients may be mixed prior to administration.

Compositions of the invention can be formulated as neutral or salt forms. Pharmaceutically acceptable salts include those formed with free amino groups such as those derived from hydrochloric, phosphoric, acetic, oxalic, tartaric acids etc., and those formed with free carboxyl groups such as those derived from sodium, potassium, ammonium, calcium, ferric hydroxides, isopropylamine, triethylamine, 2-ethylamino ethanol, histidine, procaine, etc.

In an embodiment, a solid form pharmaceutical composition is provided (e.g. tablets, capsules, powdered, or pulverized form) comprising a crystalline or amorphous gastrin compound.

In another embodiment, a solid form pharmaceutical composition is provided (e.g. tablets, capsules, powdered, or pulverized form) comprising a crystalline or amorphous gastrin compound.

In another embodiment, the invention relates to a liquid drug formulation comprising pharmaceutically acceptable salts of a gastrin compound, and to lyophilized drug formulations that can be reconstituted to provide suspensions that are stable and suitable for parenteral administration.

In a particular embodiment, the invention relates to an aqueous composition comprising pharmaceutically acceptable salts of a gastrin compound, and a solvent system which effects solubilization. The invention also provides a drug comprising an aqueous formulation of pharmaceutically acceptable salts of a gastrin compound with at least one solubilizer.

A composition of the invention may be sterilized by, for example, filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

In addition to the formulations described herein, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt.

The compositions of the invention and components thereof may comprise soluble polymers as targetable drug carriers.

After pharmaceutical compositions have been prepared, they can be placed in an appropriate container and labelled for treatment of an indicated condition. For administration of a composition of the invention, such labelling would include amount, frequency, and method of administration.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the manufacture, use or sale of pharmaceuticals or biological products, which notice reflects approval by the agency of manufacture, use, or sale for human administration.

According to another aspect of the invention, a kit is provided. In an aspect, the kit comprises a gastrin compound or a pharmaceutical composition. The kit is a package which houses a container which contains a composition of the invention and also houses instructions for administering the composition to a subject.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Effects of Gastrin or EGF on Fasting Blood Glucose and Pancreatic Insulin Content in NOD Mice with Recent Onset Diabetes

The purpose of this experiment was to determine whether gastrin (G1) or an BGF (E1) alone can improve diabetic conditions in NOD mice with recent onset diabetes.

Non-obese diabetic (NOD) female mice with chronic insulin-dependent diabetes were purchased from Taconic (Germanton, N.Y.). The mice were housed and fed under pathogen-free conditions and were cared for according to the Canadian Council on Animal Care guidelines. The NOD mice were monitored for diabetes development (fasting blood glucose, FBG>6.6 mmol/l). After the onset of diabetes, the mice were treated with (i) vehicle (V)—saline phosphate buffer (n=4), (ii) E1 (B) 1 μg/kg/day, given i.p. once daily (n=5) for 14 days, (iii) G1 (G) 3 μg/kg/day, given i.p. once daily (n=5) for 14 days. The mice did not receive insulin-replacement treatment. Fasting blood glucose levels and pancreatic insulin levels were monitored. E1 is a 51 amino acid EGF analog; G1 is a gastrin analog that is the same length as the native gastrin but contains a single amino acid change at position 15.

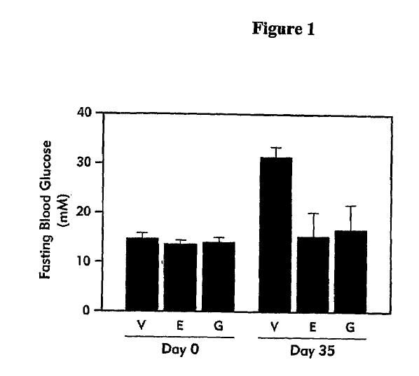

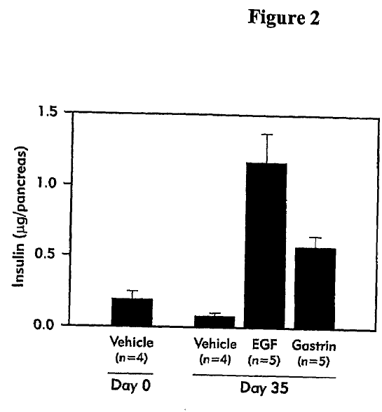

In the vehicle-treated control animals, fasting blood glucose (FBG) levels were doubled after 35 days. FBG levels of animals treated with either E1 or G1 remained close to values recorded at diabetes onset (day 0), in spite of disease progression (e.g. ongoing destruction of islet cells) in this animal model. See FIG. 1. Islet cell neogenesis stimulated by EGF or gastrin compensates for the destruction of these cells. In addition, pancreatic insulin levels were also measured in all animals. Pancreatic insulin levels for vehicle-treated controls decreased at day 35 due to destruction of β-cells, whereas animals treated with E1 or G1 exhibited significantly elevated levels of pancreatic insulin levels in comparison to the pretreatment values. See FIG. 2. The results of this study demonstrate that a short course (14 days) of treatment with either E1 or G1 after recent onset of diabetes in NOD mice can prevent progression of diabetic conditions as well as improve pancreatic insulin content for at least 3 weeks after therapy is stopped.

### Example 2

### Prolonged Effect of Gastrin on Fasting Blood Glucose in NOD Mice With Recent Onset Diabetes

The purpose of this experiment was to determine whether treatment with gastrin produced long term improvement of diabetic conditions in NOD mice with recent onset diabetes. NOD mice were purchased from Taconic (Germanton, N.Y.). The mice were housed and fed under pathogen-free conditions and were cared for according to the Canadian Council on Animal Care guidelines

Following an initial 18 day period of treatment with 3 μg/kg/day of gastrin, non-obese diabetic (NOD) mice were monitored for fasting blood glucose (FBG) levels for up to 8 weeks from the initiation of therapy. A control group of mice treated with vehicle was also monitored.

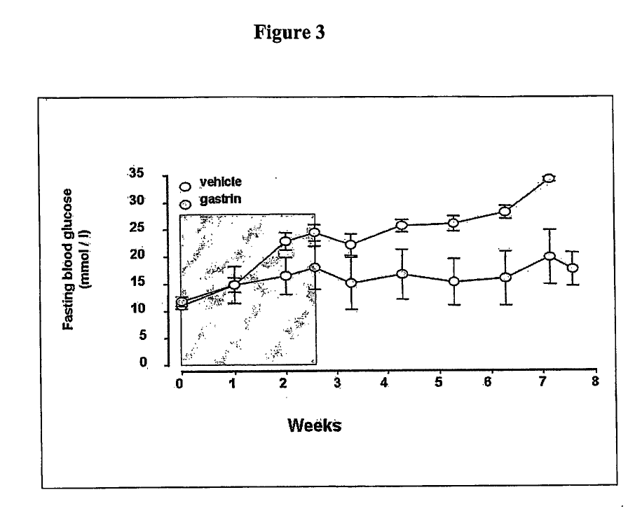

In the vehicle-treated control animals, fasting blood glucose (FBG) levels increased over time. FBG levels of animals treated with gastrin remained decreased relative to the control animals for at least 5 weeks after cessation of treatment. See FIG. 3. The results of this study demonstrate that treatment with gastrin reduces fasting blood glucose levels in NOD mice for at least 5 weeks after therapy is stopped.

### Example 3

### Dose-Dependent Effects of Gastrin on Fasting Blood Glucose in NOD Mice with Chronic Insulin-Dependent Diabetes

### Objective:

To determine whether gastrin (G1) can prevent development of severe hyperglycemia and death in NOD mice with chronic insulin-dependent diabetes.

### Method:

NOD mice with chronic insulin-dependent diabetes and maintained on insulin therapy were distributed into different treatment groups treated with: (i) vehicle (n=4); (ii) G1 1 μg/kg/day, given i.p. twice daily (n=4) for 28 days, (iii) G1 5 μg/kg/day, given i.p. twice daily (n=4) for 28 days, (iv) G1 10 μg/kg/day, given i.p. twice daily (n=4) for 28 days. Insulin therapy was stopped 14 days after commencement of treatment with G1. G1 is a 17 aa gastrin analog that is the same length as the native gastrin molecule but contains a single amino acid change at position 15.

### Results and Conclusions:

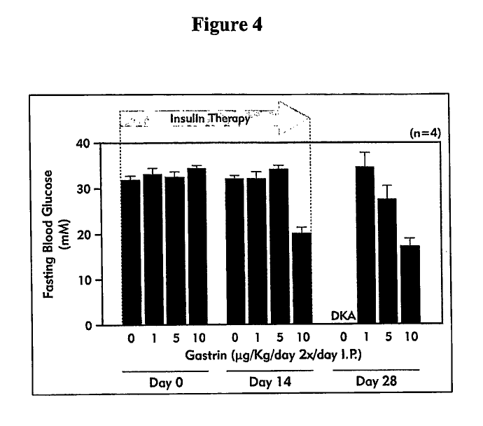

From day 0 to day 14, where the animals were maintained on insulin therapy, fasting blood glucose (FBG) levels for all treatment groups remained close to levels recorded at day 0 except for the group treated with 10 μg/kg/day of G1 which exhibited a decrease in FBG. At day 28, 14 days after the cessation of insulin therapy, all animals in the vehicle group died from diabetic ketoacidosis (DKA) since these mice were completely dependent on insulin injections. However all mice treated with G1 survived without insulin treatment for 2 weeks. Fasting blood glucose levels for mice treated with 1 μg/kg/day of G1 remained elevated but there was a corresponding decrease of fasting blood glucose levels with increasing dose of G1 (5 and 10 μg/kg/day, respectively). See FIG. 4. These data suggest that treatment with gastrin has the ability to significantly improve glucose control without the use of insulin therapy in chronically diabetic insulin-dependent NOD mice.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

1. A pharmaceutical composition comprising a therapeutically effective amount of a gastrin compound that provides sustained beneficial effects and a pharmaceutically acceptable carrier, excipient, or vehicle, wherein the therapeutically effective amount is sufficient to decrease requirements for insulin in a diabetic subject relative to requirements for insulin in the subject when the gastrin compound is not administered.

2-9. (canceled)

10. A pharmaceutical composition as claimed in claim 1 wherein the therapeutically effective amount is sufficient to increase pancreatic insulin levels by at least about 0.05%, 0.1%, 0.5%, 1%, 2%, 5%, 10%, 15%, 20%, 30%, 33%, 35%, 40%, 45%, or 50%.

11. A pharmaceutical composition as claimed in claim 1 wherein the therapeutically effective amount is sufficient to decrease blood glucose levels by at least about 2%. 5%, 10%, 15%, 20%, 30%, 40%, 50%, 60%, 70%, 80%, or 90%.

12. (canceled)

13. A method for treating diabetes in a subject comprising administering to the subject a therapeutically effective amount of at least one gastrin compound to produce a sustained beneficial effect wherein the sustained beneficial effect is decreased requirements for insulin relative to requirements for insulin in the absence of administration of a gastrin compound.

14-21. (canceled)

22. A method of claim 13 wherein the amount of gastrin compound is sufficient to provide a prolonged increase in proliferation of islet precursor cells in pancreatic tissue in the subject.

23. A method of claim 13 wherein the amount of gastrin compound is sufficient to provide a prolonged increase in the number of pancreatic insulin secreting β cells in the subject and wherein the method further comprises determining the amount of islet neogenesis.

24. A method of claim 23, wherein the amount of islet neogenesis is measured by a parameter selected from the group of: blood glucose, serum glucose, blood glycosylated hemoglobin, pancreatic β cell mass, serum insulin, pancreatic insulin content, and morphometrically determined β cell mass.

25. A method of claim 24, wherein blood glucose is reduced in the subject for a prolonged period compared to blood glucose assayed prior to administering a gastrin compound.

26. A method of claim 25, wherein blood glucose is reduced in the subject by 50% compared to blood glucose assayed prior to administering a gastrin compound.

27-28. (canceled)

29. A method of claim 23, wherein pancreatic insulin concentration is increased for a prolonged period compared to pancreatic insulin concentration in a mammal assayed prior to administering a gastrin compound.

30. (canceled)

31. A method of claim 23, wherein the gastrin compound is provided in an amount sufficient to induce prolonged differentiation of the pancreatic islet precursor cells into glucose responsive insulin secreting islet cells.

32-33. (canceled)

34. A method of claim 23, wherein gastrin compound(s) is provided in an amount sufficient to provide a prolonged increase in proliferation of pancreatic islet precursor cells.

35. (canceled)

36. A method for inducing pancreatic islet neogenesis in a mammal, the method comprising administering a composition comprising at least one gastrin compound in an amount sufficient to provide a prolonged increase in the number of pancreatic insulin secreting β cells in the mammal and to reduce requirements for insulin in the subject relative to requirements for insulin in the absence of administration of gastrin compound(s).

37-40. (canceled)

41. A kit form of a composition as claimed in claim 1.

42. A method according to claim 13 wherein at least one gastrin compound is a gastrin comprising a sequence of any one of SEQ ID NOs. 1-5 or having 60%, 70%, 80%, 90%, 95%, 98%, or 99% amino acid sequence identity with a sequence of SEQ ID NOs. 1-5.

43. A method according to claim 13 wherein at least one gastrin compound is a peptide or non-peptide agonist or partial agonist of the gastrin receptor.

44. A method according to claim 13 wherein at least one gastrin compound is a compound that increases the secretion of endogenous gastrins or is a gastric releasing peptide.

45. A method according to claim 13 wherein at least one gastrin compound is a cholecystokinin.

46. A method according to claim 13 wherein at least one gastrin compound is Omeprazole.

47. A method according to claim 13 wherein a gastrin and a gastric releasing peptide are administered.


SEQUENCE LISTING (5 sequences)

SEQ ID NO: 1 - protein, 34 aa, Artificial
  Synthesized Peptide
  MISC_FEATURE (1)..(1): Xaa = pyroglutamate
XLGPQGPPHLVADPSKKQGPWLEEEEEAYGWMDF

SEQ ID NO: 2 - protein, 34 aa, Artificial
  Synthesized Peptide
  MISC_FEATURE (1)..(1): Xaa = pyroglutamate
XLGPQGPPHLVADPSKKQGPWLEEEEEAYGWLDF

SEQ ID NO: 3 - protein, 17 aa, Artificial
  Synthesized Peptide
  MISC_FEATURE (1)..(1): Xaa = pyroglutamate
XGPWLEEEEEAYGWMDF

SEQ ID NO: 4 - protein, 17 aa, Artificial
  Synthesized peptide
  MISC_FEATURE (1)..(1): Xaa = pyroglutamate
XGPWLEEEEEAYGWLDF

SEQ ID NO: 5 - protein, 101 aa, Homo sapiens
  ref <308>: DATABASE ACCESSION NUMBER: P01350
  ref <309>: DATABASE ENTRY DATE: 2004-06-15
  ref <313>: RELEVANT RESIDUES: (1)..(101)
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN